(19) United States
(12) Patent Application Publication
(10) Pub. No.: US 2020/0397670 A1
(43) Pub. Date: Dec. 24, 2020

(54) OXYGEN GENERATING FACE MASKS

(71) Applicant: Theracell, Inc., Sherman Oaks, CA (US)

(72) Inventors: Andrew J. Carter, Sherman Oaks, CA (US); Melissa Jochim, Sherman Oaks, CA (US); Bradley E. Patt, Sherman Oaks, CA (US); Ali Hussain, Sherman Oaks, CA (US)

(21) Appl. No.: 16/975,543

(22) PCT Filed: Feb. 26, 2019
(86) PCT No.: PCT/US2019/019692

§ 371 Date: Aug. 25, 2020

Related U.S. Application Data
(60) Provisional application No. 62/635,465, filed on Feb. 26, 2018.

(51) Int. Cl. A61K 8/02; A61M 35/00; A61K 8/19; A61K 8/22; A61K 8/73; A61K 8/85; A61K 8/21; A61K 8/98; A61K 9/00; A61K 47/36; A61K 47/34; A61K 47/02; A61K 47/46; A61K 33/00; A61K 33/40

(52) U.S. Cl. CPC A61K 8/0212; A61K 33/40; A61M 35/30; A61K 8/19; A61K 8/22; A61K 8/735; A61K 8/85; A61K 8/21; A61K 8/981; A61K 9/0014; A61K 47/36; A61K 47/34; A61K 47/02; A61K 47/46; A61K 33/00; A61M 35/10

(57) ABSTRACT

A composition for healing or improving skin includes a fabric or material impregnated with an oxygen generating material. The fabric may include a first side in contact with the skin for providing oxygen to the skin and a second side between the oxygen generating material and the air surrounding the skin to form a barrier for retaining heat and inhibiting evaporation.

### CROSS-REFERENCE TO RELATED APPLICATION(S)

The present application claims priority to and the benefit of U.S. Provisional Application Ser. No. 62/635,465 filed on Feb. 26, 2018, entitled “OXYGEN GENERATING FACE MASKS,” the entire content of which is incorporated herein by reference.

### FIELD

The present disclosure generally relates to devices used in cosmetics to help overcome reduced oxygen supply in the skin. These devices supply and deliver molecular oxygen and other active nutrients into aged skin.

### BACKGROUND

Skin undergoes aging due primarily to intrinsic aging and extrinsic aging factors. The effects of intrinsic aging are caused primarily by internal factors. Also referred to as chronological aging, intrinsic aging is an inherent degenerative process due to declining physiologic function and declining capacity. This declining capacity encompasses qualitative and quantitative changes including diminished or defective synthesis of collagen and elastin in the dermis. As skin ages, it becomes thinner and more easily damaged. This effect is intensified with a decrease in the aging skin's ability to heal itself. Skin aging is also noted by a decrease in volume and elasticity and an increase in wrinkles. Aging skin receives less blood flow and lower glandular activity and therefore receives a reduced supply of oxygen and other nutrients. Cortisol (associated with stress) causes degradation of collagen, thereby accelerating the aging process. Extrinsic aging of skin is caused by external factors such as ultraviolet (UV) radiation, cigarette smoking, and air pollution. Of all extrinsic causes, radiation from sunlight has the most widespread documentation of negative effects on the skin. Because of this, extrinsic aging is often referred to as photoaging and is defined as changes to the skin caused by chronic exposure to UV light. Photodamage—i.e., damage from the sun—implies changes beyond those associated with aging alone. As such, photoaging renders two main concerns: i) an increased risk for skin cancer, and ii) the appearance of damaged skin. In younger skin, sun damage (will heal faster since the cells in the epidermis have a faster turnover rate, while in older adults, thinner skin and slower healing may result in damage to the dermal layer. Oxygen and specifically reduced oxygen supply in aged skin is implicated in the reduction of the ability of skin to heal itself from the extrinsic aging factors.

The specific processes that demand oxygen supply include extracellular matrix collagen production, elastin proliferation, general cell metabolism amongst others, all of which are necessary for sustaining healthy, toned, and elastic non-aged appearing skin. Unfortunately, the concentration of oxygen in skin is reduced with age due to compromised vasculature as a result of aging, pollution, sun exposure, smoking, alcohol use, or health related impediments the risk for which increases with age. Additionally, with age the ability of hemoglobin to concentrate oxygen diminishes resulting in a lower partial pressure of oxygen. Accordingly, oxygen is a prerequisite for healthy skin due to the increased demand for processes such as cell proliferation, bacterial defense, angiogenesis, and collagen synthesis. Furthermore, several approaches have been taken to attempt to improve oxygen delivery to skin including the use of hyperbaric oxygen therapy (HBOT). It has been reported that the superficial surface of the skin (e.g., up to 0.5 mm in depth) absorbs oxygen not only through vascular blood supply but also from the oxygen in the air.

Cosmetic formulations have been developed to improve the appearance of the skin by using various active ingredients and nutrients that may delay and reverse the signs of aging in the skin making the skin look healthier.

### SUMMARY

In some embodiments of the present disclosure, a cosmetic device for cosmetic treatment of skin is provided in which the cosmetic device (e.g., a mask) is applied to the face or other parts of the body. Some embodiments of the present disclosure include processes for making such devices.

Some embodiments of the present disclosure include methods for treating the skin to inhibit or decrease the effects of skin aging.

Some embodiments of the present disclosure include improved methods for treatment of damaged skin, such as skin that has been treated, traumatized, and/or damaged, for example, by laser, by exfoliation (e.g., chemical or mechanical), burned, and/or exposed to a harsh environment. Additional examples of damage include shaving, waxing, positive pressure or negative pressure, acne, and baldness.

Devices according to embodiments of the present disclosure include a nonwoven fiber layer that acts as a carrier for an oxygen generating material that when wetted decompose to liberate oxygen. Examples of an oxygen generating material include calcium peroxide. The wetted device is placed against the skin to deliver the liberated oxygen to the skin.

Embodiments of the present disclosure include devices where the wetting process includes contacting the oxygen carrier impregnated layer with a perfluorocarbon (PFC) emulsion or gel to provide a more effective means of providing a reservoir of oxygen, and providing oxygen transport to the skin.

In some embodiments of the present disclosure, methods of treating the skin may be carried out over prolonged time periods using a dressing in which the presently disclosed oxygen generating material is encapsulated in a resorbable polymer to control the release of oxygen over a period of time (e.g., multiple days) during which the dressing is applied to the skin.

In some embodiments of the present disclosure, methods of treating the skin may be carried out over shorter time periods (e.g., less than 1 day, 1-3 hours, or less than 1 hour) using a face mask in which the oxygen generating material is encapsulated in a water soluble carrier in the face mask.

In some embodiments of the present disclosure, a PFC emulsion may contain other factors designed to improve skin health and function.

In some embodiments of the present disclosure, the impregnated nonwoven may contain other dry ingredients that when wetted form a cosmetic cream, or may contain other factors designed to improve skin health and function.

In some embodiments of the present disclosure, the mask materials form a heat barrier.

In some embodiments of the present disclosure, devices for treating skin with a cream include a film laminated to the upper surface or a cover film to act as a barrier to inhibit oxygen and/or moisture loss from the treatment into the atmosphere, to facilitate handling, and/or to inhibit the cream coming into contact with clothing or skin not requiring treatment.

Devices according to embodiments of the present disclosure may also be in the form of a first aid dressing or a bandage (e.g., Band Aid®), or they may be used in conjunction with an adhesive film dressing such as OpSite (Smith & Nephew) or Tegaderm™ (3M).

### BRIEF DESCRIPTION OF THE DRAWINGS

FIG. 1 shows a device according to embodiments of the present disclosure, the device having an oxygen barrier layer (1) and a nonwoven layer (2) containing an oxygen generating material.

FIG. 2 is a schematic of a face mask according to embodiments of the present disclosure, where the nonwoven containing oxygen generating material (2) has cut outs (3) for the users eyes, (4) for breathing, and (5) for the mouth, where the device as shown may also have cut outs (6) positioned to facilitate draping and conforming of the device to the face or other part of the body.

FIG. 3 is a schematic of a face mask having two halves that are folded onto each other along a fold (8) in which the mask is activated upon contact of one half with the other half, according to embodiments of the present disclosure. As indicated in the schematic, the right hand side of the device is a nonwoven (2) containing an oxygen generating material and the left hand side (9) is constructed with a PFC emulsion that may also be on a carrier.

FIG. 4 is a schematic of a bandage strip (e.g., Band-Aid®) type of device where the oxygen barrier material (1) forms the backing layer, the nonwoven layer with oxygen generator material (2) forms the pad, and there is a pressure sensitive adhesive (10) designed to hold the device in place, according to embodiments of the present disclosure.

FIG. 5 is a graph of oxygen release data over time from example face masks, as indicated, according to embodiments of the present disclosure.

FIG. 6 is a graph oxygen release data over time from oxygen generator materials described in Examples 9 and 10 as indicated, according to embodiments of the present disclosure.

### DETAILED DESCRIPTION

Compositions according to embodiments of the present disclosure include creams, serums, emulsions, and gels having high gas solubility compounds capable of carrying oxygen (e.g., perfluorocarbons (PFCs)) for transporting oxygen to the skin to increase and/or improve the oxygen content in the skin. Compositions of the present disclosure which are infused with an oxygen carrier may be applied to a device (e.g., a material) including a bandage or a non-woven that may be applied to skin as a dressing for administration of the oxygen carrying composition to the skin. Accordingly, compositions and devices of the present disclosure provide oxygen and other active ingredients to inhibit or treat the effects of aged and/or damaged skin.

Compositions and devices as presently disclosed are based on studies on the delivery of oxygen to the skin that have shown dissolved topical oxygen will penetrate skin more effectively than gaseous oxygen. Roe et al., Journal of Surgical Research, 2010, 159:29-36, the entire content of which is incorporated herein by reference. Roe et al. also reported that transcutaneous penetration of topically applied dissolved oxygen was shown to penetrate through >700 μm of human skin. For reference, the epidermis is approximately 100 μm (micrometers or microns) in thickness and the dermis is approximately 2 mm (millimeters) in thickness. Furthermore, a topically applied dissolved oxygen dressing was reported to be well tolerated with several measures of skin health and integrity showing improvements compared with a control dressing. Kellar et al., Journal of Cosmetic Dermatology, 2013, 12:86-95, the entire content of which is incorporated herein by reference.

Because the skin is most often at an elevated temperature compared with the surrounding environment, the active ingredients in a composition to be applied to the skin may readily evaporate from the surface of the skin. Moreover, oxygen carriers such as PFCs and other active ingredients being deployed beneficially to the skin are large molecules which are not easily absorbed into the skin. To address these issues, the devices and materials according to embodiments of the present disclosure include material sheets embedded with an oxygen carrier on a first side of the material that will come in contact with the skin with the second side of the material forming a barrier, thereby trapping the oxygen carrying composition on the skin to facilitate effective absorption into the dermis of the skin.

As used herein, the term “device” refers to a composition to be applied to the skin on which the oxygen carrying composition may be incorporated. The device may include demineralized bone fibers (DBF) and other examples as described in more detail in this disclosure. The device may also include “material sheet,” “sheets of material,” “masks,” “face mask,” “sheet masks,” or “fabric,” all of which refer to any suitable material as disclosed herein, to receive or incorporate the oxygen carrier to be provided to the skin to be treated. Non-limiting examples of materials from which these sheet masks or fabrics are made include: wovens, non-wovens, foil laminations, bio-cellulose layers, hydrogel, aquatic technology, rubber, and other materials suitable for forming a barrier between a formulation applied to the skin and the environment. As disclosed herein, these materials may function as heat barriers to reduce evaporation and increase retention of the heat that would otherwise radiate from the skin. Moreover, heat retention may also help skin cells absorb the active ingredients in the composition on the sheet mask.

Aspects of embodiments of the present disclosure are directed to cosmetic masks made from a fabric, including face or body masks, to deliver oxygen, dissolved oxygen, molecular oxygen, and/or oxygen based cosmetic formulations to the skin.

As used herein, fabric refers to a woven or non-woven material. A woven material is a cloth formed by weaving, and a nonwoven material is made from staple fibers and/or long fibers bonded together by chemical, mechanical, heat or solvent treatment.

In the following disclosure, certain exemplary embodiments of the present disclosure are shown and are described by way of illustration. As those skilled in the art would recognize, the described exemplary embodiments may be modified in various ways without departing from the spirit or scope of the present disclosure. Accordingly, the drawings and descriptions are to be regarded as illustrative in nature, and not restrictive.

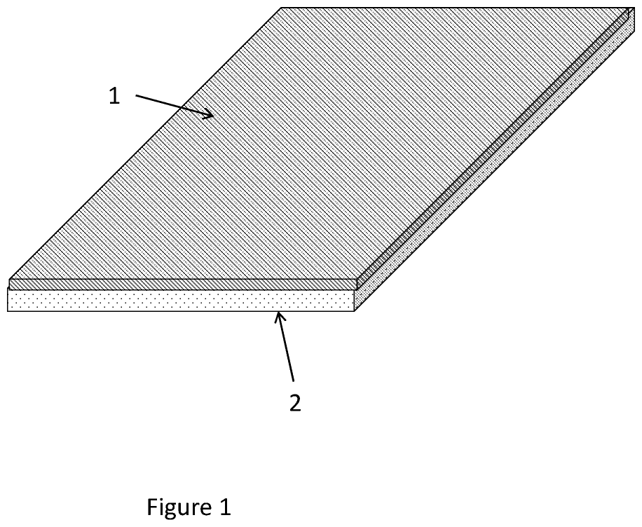

With reference to FIG. 1, a rectangular device according to embodiments of the present disclosure includes a nonwoven layer (2) that acts as a carrier for an oxygen generating material, and a layer (1) that acts to direct oxygen towards the skin. The device is wetted with water prior to placing on the skin, and once wetted the device becomes conformable and may be smoothed into place.

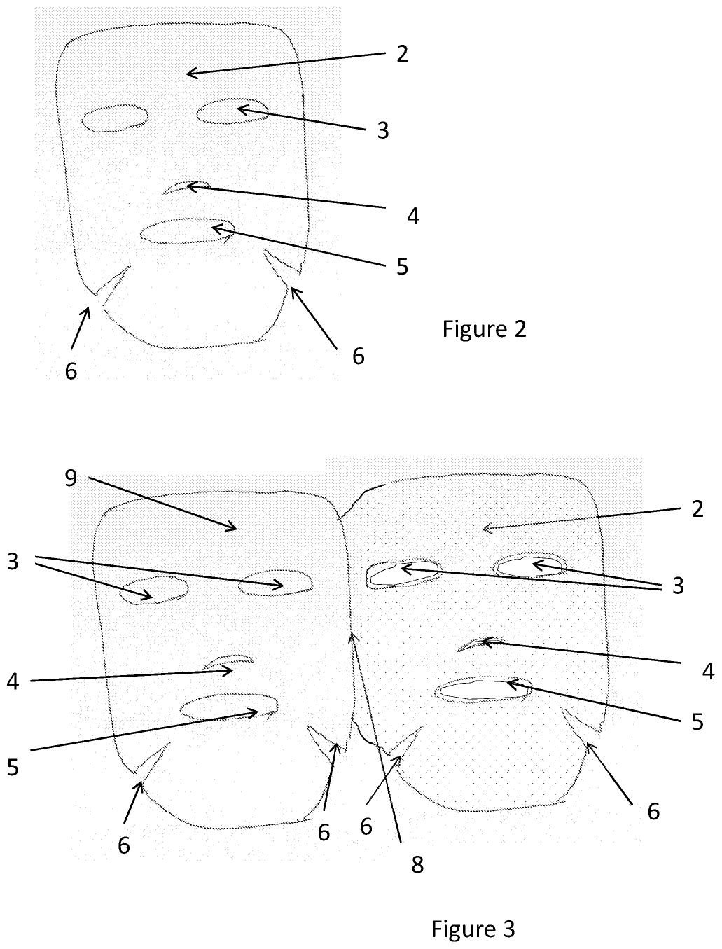

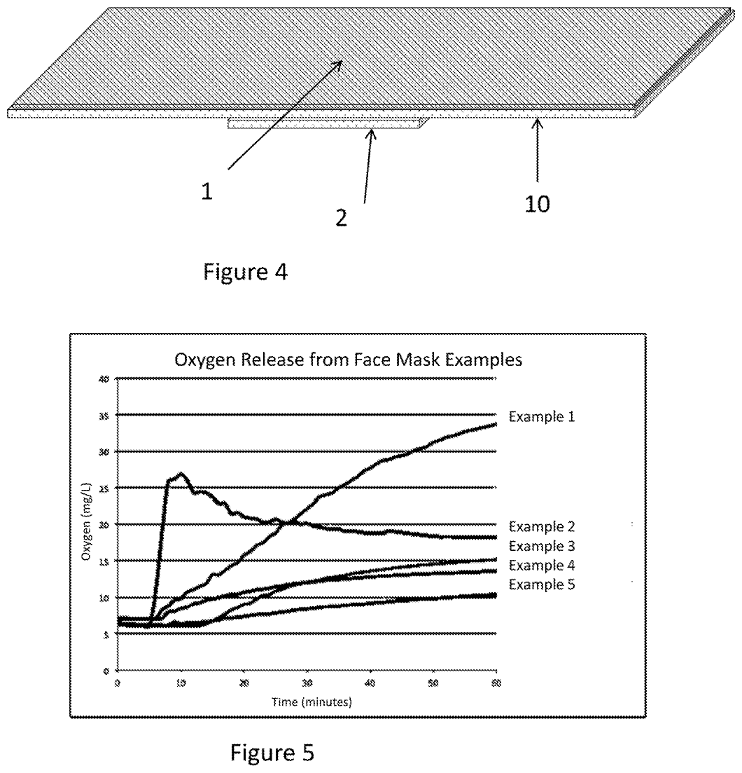

With reference to FIG. 2, the functional layers of the treatment as described in FIG. 1 are organized into a mask capable of being fit over a face for cosmetic skin treatment. Once wetted, the face mask may be applied onto the face and left in place for a few minutes, up to a few hours, or overnight. The device has cut outs (3) for the eyes, (4) for the nose and (5) for the mouth. The device also has optional cuts outs (6) that help facilitate conformance with the face. While in some embodiments the mask includes an oxygen generating material impregnated nonwoven, in other variants there the mask may also include an oxygen barrier material (1). In addition to improving the direction of oxygen into the skin, this barrier layer may also serve to prevent the cream from the mask soiling clothing or bedding. While the mask of FIG. 2 is cut into a shape to fit a face, this mask may be applied to any part of the body, and the mask material may be shaped into any suitable form to be applied to other areas of skin on the body.

With reference to FIG. 3, the right hand side of the face mask device may be a nonwoven (2) containing an oxygen generating material and the left hand side (9) may include a PFC emulsion that may also be on a carrier. Additionally, packaging of the two device (mask) halves may be designed to keep the two halves isolated from each other and from moisture. The right hand side of the device may also have an oxygen barrier layer, arranged such that this would be the outermost layer of the device and the PFC emulsion side would be the skin contacting side.

The oxygen barrier material for a face mask may be fabricated from a polymer with low oxygen permeability such as Poly(ethylene vinyl alcohol), or may be a metallic foil.

The nonwoven material is a fibrous conformable material that acts as a carrier for the oxygen generating materials as disclosed herein.

Suitable nonwovens may be fabricated using polyester, cotton, polypropylene, polyethylene, blends thereof, and/or other synthetic or natural fibers, and may be selected to be conformable to the skin and to have a high surface area to facilitate retention of the oxygen generating materials.

As those skilled in the art will realize the nonwoven material may be substituted with any other fibrous/high surface area conformable material such as a knitted or woven fabric, or a foam.

In some embodiments of the present disclosure, the nonwoven material is formed from demineralized bone fibers (DBF™). Such materials are disclosed in U.S. Pat. Nos. 9,486,557 and 9,572,912. The use of a material that is an extracellular matrix allows the many beneficial growth factors to be eluted from the matrix during the device application. The growth factors promote regeneration, healing and activation of various cellular cascades that improve skin health including hair follicle generation.

Oxygen Generating Materials. Oxygen is generated by the breakdown of materials such as Calcium Peroxide, Magnesium Peroxide, Sodium Percarbonate, or Sodium Peroxide. In some instances, Hydrogen Peroxide may be an intermediate product that may require catalysis for it to break down. Catalysts such as catalase or zinc oxide can be used.

The oxygen generator may be incorporated into a nonwoven, or other fibrous carrier by use of a water-soluble binder. To prevent premature breakdown, the generator/carrier mixture will need to be impregnated or coated onto the nonwoven using a non-aqueous solvent.

Water soluble binders include polyoxamers, polyvinyl pyrrolidones, polyvinyl alcohol, carboxy methyl cellulose, polyacrylates, polyethylene oxide, gelatin, hydroxyl propyl cellulose, polyethylene glycol, polyacrylic acid, polyacylamides, N-(2-hydroxypropyl) methacrylamide, Divinyl ether-maleic anhydride, polyoxazolines, xanthum gum, pectins, dextran,

For longer term release, the oxygen generator material is encapsulated in a resorbable polymer to affect control over the rate of water exposure to the oxygen generator and hence control the rate of oxygen generation.

Resorbable polymers that may be used to encapsulate the oxygen generator include, but are not limited to, proteins, including silk, collagen (including Types I to V and mixtures thereof), and proteins including one or more of the following amino acids: alanine, arginine, asparagine, aspartic acid, cysteine, glutamine, glutamic acid, glycine, histidine, isoleucine, lysine, methionine, phenylalanine, proline, serine, threonine, tryptophan, tyrosine and valine; polysaccharides, including alginate, amylose, carboxymethylcellulose, cellulose, chitin, chitosan, cyclodextrin, dextran, dextrin, gelatin, gellan, glucan, hem icellulose, hyaluronic acid, derivatized hyaluronic acid, oxidized cellulose, pectin, pullulan, sepharose, xanthan and xylan; resorbable polyesters, including resorbable polyesters made from hydroxy acids (including resorbable polyesters like poly(lactides), poly(glycolides), poly(lactide-co-glycolides), poly(lactic acid), poly(glycolic acid), poly(lactic acid-co-glycolic acid), poly(dioxanones), polycaprolactones and polyesters with one or more of the following monomeric units: glycolic, lactic; trimethylene carbonate, p-dioxanone, or ε-caprolactone), and resorbable polyesters made from diols and diacids; polycarbonates; tyrosine polycarbonates; polyamides (including synthetic and natural polyamides, polypeptides, and poly(amino acids)); polyesteramides; poly(alkylene alkylates); polyethers (such as polyethylene glycol, PEG, and polyethylene oxide, PEO); polyvinyl pyrrolidones or PVP; polyurethanes; polyetheresters; polyacetals; polycyanoacrylates; poly(oxyethylene)/poly(oxypropylene) copolymers; polyacetals, polyketals; polyphosphates; (phosphorous-containing) polymers; polyphosphoesters; polyalkylene oxalates; polyalkylene succinates; poly(maleic acids); biocompatible copolymers (including block copolymers or random copolymers); and hydrophilic or water soluble polymers, such as polyethylene glycol, (PEG) or polyvinyl pyrrolidone (PVP), with blocks of other biocompatible or biodegradable polymers, for example, poly(lactide), poly(lactide-co-glycolide), or polycaprolactone or combinations thereof. Resorbable polymers also include cross-linked polymers, and include, for example, cross-linked collagen, as well as functionalized polymers. Particularly preferred resorbable polymers resorbable polyesters.

In an alternative embodiment the carrier material is formed into a foam such that it takes on the role of the nonwoven.

In an alternative embodiment the oxygen generator is produced in the form of a fiber. Use of a core-sheath fiber system with high load of the oxygen generator in the core allows ability to protect the system to give storage stability and to control the rate of oxygen generation. It also allows the physical properties of the fiber to be dictated by the sheath. The core-sheath fiber system can be generated by electrospinning, or wet spinning using a double lumen needle.

A porogen such as calcium carbonate may also be included in the outer sheath to provide buffering capacity to help maintain a physiologic pH in instances where the breakdown of the oxygen generator leads to a lowering of the pH. Alternatively or additionally, buffering agents and catalysts may be formulated into the oxygen generating core material.

Alternatively or additionally, the resorbable polymer/oxygen generator material may be formed as microspheres. Both microspheres and fibers may be suitably formed by precipitation of the solvent polymer oxygen generator mixture into a non-solvent.

Alternatively or additionally, the solvent, polymer, oxygen generator may be dried to remove the solvent and then ground to a powder.

The controlled release fibers or spheres may then be coated or impregnated into the nonwoven using a water-soluble polymer or binder. To avoid prematurely activating the oxygen generator the coating, impregnation should be undertaken using non aqueous solvents. The coating/impregnation may cover all of the nonwoven, all of one side of the nonwoven, or it may be patterned as can be achieved, for example, by screen printing or by using a modified ink jet printer.

Alternatively or additionally, the polymer oxygen generator mixture may be impregnated directly into the nonwoven.

The impregnate may optionally also include additional materials to provide for other benefits in the product. For example, for a cosmetic treatment ingredients may include, but are not limited to, and may include mixtures of botanical extracts with antioxidant, soothing, calming, anti-wrinkle, clarifying, nutrient and other properties such as: acai berry (Euterpe olerace) extract, arnica (Arnica montana) extract, bamboo (Bambusa vulgaris) extract, calendula (Calendula officinalis) extract, chamomile (Chamomilla recutita) extract, cucumber (Cucumis sativus) extract, Ginkgo biloba extract, green tea (Camellia sinensis) extract, horse chestnut (Aesculus hippocastanum) extract, pepper tree (Schinus terebinthifolius) seed extract, willow bark (Salix alba) extract and witch hazel (Hamamelis virginiana) extract.

The impregnate may optionally also include ingredients that provide calming and soothing to the skin by including ingredients such as but not limited to, and may include mixtures of algae, algin, sodium alginate, kelp (Laminaria digitata) extract, allantoin, aloe vera (aloe barbadensis), bisabolol and sodium hyaluronate.

The impregnate may optionally also include ingredients that provide plant oils rich in skin nutrients and essential fatty acids by including ingredients such as but not limited to, and may include mixtures of argan (Argania spinsosa) oil, borage (Borago officinalis) oil, coconut (Cocos nucifera) oil, cranberry (Vaccinium macrocarpon) oil, evening primrose (Oenothera biennis) oil, flaxseed (Linum usitatissimum) oil, grape seed (Vitis vinifera) oil, jojoba (Simmondsia chinensis) oil, lavender (Lavandula officinalis) oil, meadowfoam (Limnanthes alba) oil, olive (Olea europaea) oil, pomegranate (Punica granatum) oil, rosehip (Rosa canina) oil, shea butter (Butyrospermum parkii) and sunflower (Helianthus annuus) oil.

The impregnate may optionally also include ingredients that provide skin clarifying benefits for acneic skin by including ingredients such as but not limited to, and may include mixtures of bentonite clay, kaolin clay, calamine, charcoal, bromelain, papain, sulfur, adapalene and salicylic acid.

The impregnate may optionally also include ingredients that provide anti-aging benefits by including ingredients such as but not limited to, and may include mixtures of antioxidants, caffeine, dimethylaminoethanol (dmae), glycolic acid, lactic acid, malic acid, peptides (hexapeptides, pentapeptides & copper peptides), resveratrol, tranexamic acid, ubiquinone (coenzyme q10), copper gluconate, magnesium aspartate, zinc gluconate, retinyl palmitate, niacinimide (vitamin B3), vitamin C (magnesium ascorbyl phosphate), vitamin C ester and vitamin E (tocopherol acetate).

The impregnate may optionally also include ingredients designed to maintain a physiologic pH, such as citric acid and may also contain chelating agents designed to bind metal ions.

For a post-peel (e.g., laser, chemical or other), post chemical, post-exfoliation (e.g., mechanical), or post-burn, the impregnate may also include but is not limited to, and may include mixtures of calming agents such as aloe vera which is rich in vitamins and minerals and which moisturizes, soothes and helps calm inflammation, and bisabolol, which limits the release of pro-inflammatory mediators, soothing the skin, tissue regeneration factors such as heparin, fibroblast growth factors, and other tissue engineering agents, and hydrating and moisturizing agents such as sodium hyaluronate, including both high and low molecular weight sodium hyaluronate to most effectively penetrate the skin barrier allowing essential moisture and hydration to be drawn into the skin, and allantoin which increases the capacity of the skin to absorb water, calming and hydrating the skin.

For cut, shaved, waxed or abraded skin dressing the impregnate may also include but is not limited to, and may include mixtures of calming agents such as aloe vera which is rich in vitamins and minerals and which moisturizes, soothes and helps calm inflammation, and Bisabolol, which limits the release of pro-inflammatory mediators, soothing the skin, tissue regeneration factors such as heparin, fibroblast growth factors, and other tissue engineering agents, and hydrating and moisturizing agents such as sodium hyaluronate, including both high and low molecular weight hyaluronate to most effectively penetrate the skin barrier allowing essential moisture and hydration to be drawn into the skin, and allantoin which increases capacity of the skin to absorb water calming and hydrating the skin.

When the impregnation is done in a patterned manner, as described above, the optional ingredients may also be printed as separate dots of material.

At the time of use, the device is activated by wetting it with water. Alternatively, as discussed below an alternate wetting agent with beneficial properties is a PFC emulsion.

Gas Transportation and Gas Reservoirs. Materials that have an inherently high solubility for oxygen may be used as reservoirs to capture and store the generated oxygen and to facilitate its transport to the skin. Fluorocarbons (FCs) such as perfluorocarbons (PFCs) have oxygen solubilities that are approximately 20 times that of water. They are stable, non-toxic, water-immiscible gas-transporting chemicals. Thus, FCs have been used in many biomedical applications. FCs are able to dissolve gases such as oxygen, nitrogen, and carbon dioxide in quantities far in excess of other materials such as water and hydrocarbon materials. FCs are capable of dissolving and thus transporting enough oxygen for example to be considered as possible blood substitutes.

Fluorocarbons are hydrophobic in nature and immiscible with water based and organic based systems. Hence, fluorocarbons are often formulated in emulsions, dispersions and gels.

Under ambient conditions when exposed to air, the PFC's in the emulsion will contain the same ratio of gases found in air, however, saturated or supersaturated forms of the emulsions may be made by exposing the emulsion to a gas at or above ambient pressure under temperature and time conditions necessary to displace other gases in the PFC with the desired gas. For example, the PFC in the emulsion may be saturated or supersaturated by exposing the emulsion to oxygen, and the oxygen may be in the form of molecular oxygen, at pressures at or above ambient and under temperature and time conditions necessary to displace the other gases that are normally in equilibrium with ambient ratios, or by simply bubbling oxygen through the fluid.

Once the proportion of oxygen in the FC or FC emulsion has been increased the material is not stable and oxygen will be lost to the surroundings unless the material is stored in oxygen barrier packaging.

In certain embodiments it is beneficial for the FC emulsion to be stored separately from the impregnated nonwoven, which if stored dry can prevent premature oxygen generation. In one embodiment this can be accomplished by storing the emulsion in a conventional cosmetic airless dispenser. In a second embodiment the emulsion is stored in in a sachet using a material selected to provide an oxygen barrier, such as foil.

In one embodiment, at the time of use, the FC emulsion may be spread evenly over the nonwoven.

In an embodiment of the present disclosure, the coating of the FC emulsion onto the impregnated nonwoven serves three purposes. Firstly, the aqueous portion of the emulsions serves to activate the decomposition of the oxygen generating species. Secondly, the PFC acts as a high oxygen soluble reservoir to absorb the generated oxygen, and thirdly it allows diffusion of oxygen to the skin surface where it can be absorbed by the skin.

In some embodiments of the disclosure the FC emulsion is a gel that can retain its shape and is presented as a sheet of material in a foil pouch that is combined with the nonwoven sheet immediately before application.

Non-limiting examples of PFCs include perfluorodecalin, perfluorohexane, perfluoroperhydrophenanthrene, perfluorobutylamine (PFTBA or PFTBM), perfluorooctylbromide (PFOB), perfluoro-n-octane, octafluoropropane, perfluorodichlorooctane, perfluorodecalin (PFD), perfluorotripropylamine, perfluorotrimethylcyclohexane, perfluoromethyladamantane, perfluorodimethyladamantane, perfluoromethyldecaline, perfluorofluorene, diphenyldimethylsiloxane, hydrogen-rich monohydroperfluorooctane, alumina-treated perfluorooctane, mixtures thereof, or any suitable perfluoronated oxygen carrier.

In other embodiments the oxygen carrier is any suitable material that is capable of carrying oxygen to the skin.

Perfluorocarbons are extremely hydrophobic materials and as such this property makes their incorporation into wound dressing materials difficult. Embodiments of the present disclosure include methods for incorporating perfluorocarbons into the skin dressing materials as disclosed herein.

Perfluorocarbons (PFCs) may be formed into emulsions by, for example, vortexing a dispersing agent solution with the PFC. The emulsions may be thickened by the addition of a thickening agent (e.g., a water-soluble polymer) into the water phase of the emulsion. Additionally, the emulsion may be concentrated by use of a centrifuge. Non-limiting examples of dispersing agents include glycerols, phospholipids, lecithins, surfactants, and polyoxamers. Non limiting examples of thickening agents include carnauba wax, hyaluronic acid, gellan, guar gum (cyamopsis tetragonolobus), hydroxyethyl cellulose, hydroxypropyl starch phosphate, lecithin, maltodextrin, squalene, and xanthan gum.

Under ambient conditions when exposed to air, the PFCs in the emulsion may contain the same ratio of gases found in air.

Saturated or supersaturated forms of the emulsions may be made by exposing the emulsion to a gas at or above ambient pressure under temperature and time conditions necessary to displace other gases in the PFC with the desired gas. For example, the PFC in the emulsion may be saturated or supersaturated by exposing the emulsion to oxygen, and the oxygen may be in the form of molecular oxygen, at pressures at or above ambient and under temperature and time conditions necessary to displace the other gases, or by simply bubbling oxygen through the fluid.

The PFC emulsions may contain additional anti-aging ingredients that help to provide relaxation to skin muscles, diminish visible fine lines and wrinkles and help restore elasticity and suppleness. Example ingredients include but are not limited to peptides (e.g., including pentapeptides and copper peptides) and others known to those skilled in the art and combinations thereof, to reduce wrinkles and fine lines.

In some embodiments, the PFC emulsion compositions as disclosed herein may include calming and soothing ingredients. These calming and soothing ingredients are suitable for application after the skin has undergone a chemical or mechanical exfoliation treatment or laser treatment.

In some embodiments, the PFC emulsion composition includes ingredients selected to protect against the synthesis and accumulation of fat molecules that are known to cause stretch marks and cellulite. Example ingredients include but are not limited to caffeine and others as known to those skilled in the art.

In some embodiments, the PFC emulsion composition includes ingredients selected to stimulate blood flow and lymphatic circulation. Example ingredients include but are not limited to horse chestnut extract and others known to those skilled in the art.

In some embodiments, the PFC emulsion composition includes ingredients selected to facilitate cellular metabolism in the dermal layers of the skin and decrease accumulation of lipoid (fat) molecules. Example ingredients include but are not limited to ingredients such as pepper seed extract and others known to those skilled in the art.

In some embodiments, the PFC emulsion composition includes ingredients selected to calm inflammation and help alleviate bruising and mitigate scarring. Example ingredients include but are not limited to ingredients such as arnica flower extract and others known to those skilled in the art.

In some embodiments, the PFC emulsion composition includes ingredients selected to help to inhibit sun damage and even appearance of pigmentation and brighten the appearance of the skin. Example ingredients include but are not limited to ingredients such as tranexamic acid and others known to those skilled in the art.

In some embodiments, the PFC emulsion composition includes ingredients selected to hydrate and restore moisture. Example ingredients include but are not limited to those including emollients such as shea butter and sodium hyaluronate and others known to those skilled in the art.

In some embodiments, the PFC emulsion composition includes ingredients selected to help calm inflammation, redness, black heads, and puffiness and/or help alleviate dark circles for example, under the eyes. Example ingredients include but are not limited to arnica flower extract and others known to those skilled in the art.

In some embodiments, the PFC emulsion composition includes ingredients selected to provide antioxidant support helping protect the skin against free radical damage. Example ingredients include but are not limited to ester of vitamin C, camelia oleifera and others known to those skilled in the art.

In some embodiments, the PFC emulsion composition includes ingredients selected to boost collagen production and cell turnover. Example ingredients include but are not limited to retinyl palmitate a vitamin A derivative and others known to those skilled in the art.

In some embodiments, the PFC emulsion composition includes ingredients selected to increase the amount of gas production to elevate the feel of the device on the skin. Example ingredients include sodium bicarbonate.

In other embodiments the devices are placed on skin, such as the face, to provide cosmetic treatment as a face mask. This may be a short duration treatment of less than 1 hour after which the mask is removed and the residual cream gently massaged into the skin. Alternatively, the face mask may be applied overnight (e.g., for 8 to 12 hours).

Biological and/or biocompatible materials may be used to prepare the nonwoven fabric. Examples of biological materials include allogenic or xenogeneic tissues such as acellular dermal matrix materials, cell or dermal growth factor-seeded dermal matrix material or cell or dermal growth factor-seeded resorbable polymers, and small intestine submucosa. In some embodiments, bovine or human bone is demineralized and fibers formed therefrom following the methodology disclosed in U.S. Pat. Nos. 9,486,557 and 9,572,912 that are included herein in their entirety for reference. Fibers may optionally be treated with a chaotropic agent such as guanidine hydrochloride to remove bone morphogenic proteins and other materials naturally occurring within the demineralized bone fiber material. Fibers from a xenogeneic source may also be treated with α-galactosidase or similar materials to reduce possible immunological response. Fibers may also be treated with a plasticizer such as glycerol that renders the dried fiber flexible. The fibers may then be formed into a nonwoven using wet lay techniques as described in the patents and then dried. Such a nonwoven fabric would additionally deliver its endogenous growth factors to the skin or wound post activation by wetting.

Embodiments of the nonwoven fabric device made from DBF may be processed to retain the inherent growth factors from the bone. In these instances the retained growth factors include BMP2 that has been shown to stimulate hair follicle production. Such nonwoven devices may find particular utility in treating areas of the skin suffering from hair thinning or hair loss, or following skin grafting or burn treatment.

Examples of bioactive agents that may be incorporated into the material sheet devices include, but are not limited to, angiogenic factors such as butyric acid, growth factors (e.g. VEG-F), inhibitors of matrix metalloproteinases (MMPs), agents such as retinols to aid oxygen diffusion through the tissue, antioxidants such as ascorbates to ameliorate the effects of reactive oxygen species, antibiotics (including silver particles), biofilm inhibitors, vitamins, anti-inflammatory drugs, lipids, steroids, hormones, antibodies, proteins, peptides, glycoproteins, signaling ligands, platelet rich plasma, amniotic membrane materials, anti-septic agents, analgesics, anesthetics, immunomodulatory agents, molecules that promote the formation of extra cellular matrix, vascularization, and wound healing. Particularly preferred antibiotics include bacitracin, neomycin, polymixin B, zinc, fusidic acid, gentamicin, mafenide acetate, metronidazole, minocycline, mupirocin, nitrofurazone, polymixin, retapamulin, rifampin, silver particles, silver sulfadiazine, sulfacetamide, vancomycin, and combinations thereof.

Methods have been developed to produce devices that may be used to treat skin derived from allogenic or xenogeneic bone that allow the release of oxygen for the treatment of skin.

Fibers may be manufactured from demineralized bone using, for example, the methods disclosed in U.S. Pat. Nos. 9,486,557 and 9,572,912. Briefly, the bone is cut into struts that are then placed in dilute acid to effect demineralization. The demineralized struts are then cut using a blade to form ribbon like fibers that may be up to 4 cm in length or greater and 0.1 to 1.5 mm wide and 0.05 to 0.5 mm thick. The bone may be derived from human, bovine, porcine or other animal sources. For utility in soft tissue healing the fibers may optionally be treated to remove bone morphogenic proteins or other naturally present materials. Suitable methods are known in the art. For example, the fibers may be treated with guanidine hydrochloride. Fibers from a xenogeneic source may also be treated with α-galactosidase or similar materials to reduce possible immunological response. The resultant fibers may then be processed using a wet lay technique to produce a fiber sheet. Cohesion of the device is optionally improved by use of a heat treatment step, as disclosed in the patent.

In some embodiments, the fibers may be dried and then rehydrated in a glycerol solution prior to the wet lay process and dried by lyophilization afterward. This renders a flexible fiber device in the absence of aqueous hydration.

In some embodiments, other collagen matrices from soft tissue such as muscle, ligament, or skin may be incorporated into or used as the material sheet device.

In some embodiments, collagen from plants, fruits, vegetables or other biological materials may be incorporated into or used as the material sheet device.

In some embodiments, scaffolds from silk may be incorporated into or used as the material sheet device.

For longer term release, the oxygen generator material may be encapsulated in a resorbable polymer to affect control over the rate of water exposure to the oxygen generator and hence control the rate of oxygen generation. The polymer/oxygen generator material may be produced in a number of forms such as fibers or microspheres.

In some embodiments, the fibers may be formed by electro-spinning, melt, wet or solvent spinning. The nonwoven material can be formed directly or by a subsequent process such as carding and needling or point bonding.

The devices may contain foams, including open or reticulated cell foams, sponges, and other porous forms. These foams may be produced, for example, by phase-separation, melt-foaming, and particulate leaching methods. Alternatively, a film is frozen to precipitate the polymer, and the solvent sublimated using, for example, a lyophilizer, to form a phase separated porous polymeric foam.

The foams may also be produced by particulate leaching methods. Pore size and density can be controlled by selection of the leachable material, its size and quantity. Foams may be formed by dispersing particles in a solution of a permanent or resorbable polymer described above, wherein the particles do not dissolve in the solvent. The solvent is subsequently evaporated, and the particles leached away with a solvent that dissolves just the particles. The foams may also be produced by melt-foam ing using blowing agents.

The oxygen generator may be incorporated as part of the foam forming formulation or alternatively the foam can be infused with the oxygen generator using a coating or impregnation process.

The devices are placed on the skin so that the oxygen can enter the skin. The devices may incorporate adhesives to help keep the device in place, and/or the devices may be held in place by a dressing material. For example, the devices may be held in place using compression dressings, such as when the devices are used to treat damaged skin on legs. In another embodiment, the device is an island on an adhesive coated film or fabric.

The material sheet devices as disclosed herein may also be used as dressings and removed after a period of time or replaced after a short period of time. In some embodiments, the material sheet devices may be replaced or additional devices placed on the skin in order to provide continued delivery of oxygen to the skin.

Modifications and variations of the devices, processes, and methods described herein will be obvious to those skilled in the art and are intended to come within the scope of the appended claims.

The following examples and the example formulations demonstrate the oxygen release concept and may also incorporate all of the additional excipients as disclosed herein, including, for example, any preservatives or pH balancing components.

### Example 1

Polyvinyl pyrrolidone 40,000 MW was dissolved in methanol to form a 30% w/v solution. 0.7 grams (g) Sodium Percarbonate, 0.3 g of Super Low Molecular Weight Hyaluronic Acid, 0.3 grams High Molecular Weight Hyaluronic Acid and 0.3 grams of Aloe Vera were added to 5 grams of the polymer solution. The resultant mixture was impregnated into a 4 cm×4 cm polyester nonwoven fabric (RB-273-40-W/R supplied by WPT Non Wovens). The resultant material was dried in a vacuum oven with 0.5 L/min air bleed.

### Example 2

Polyvinyl pyrrolidone 360,000 MW was dissolved in methanol to form a 10% w/v solution. 0.5 grams Sodium Percarbonate, 0.2 g of Super Low Molecular Weight Hyaluronic Acid, and 0.2 grams of Aloe Vera were added to 5 grams of the polymer solution. The resultant mixture was impregnated into a 4 cm×4 cm polyester nonwoven fabric (RB-273-40-W/R supplied by WPT Non Wovens). The resultant material was dried in a vacuum oven with 0.5 L/min air bleed.

### Example 3

Polyvinyl pyrrolidone 360,000 MW was dissolved in methanol to form a 10% w/v solution. 0.5 grams Sodium perborate monohydrate, 0.2 g of Super Low Molecular Weight Hyaluronic Acid, and 0.2 grams of Aloe Vera were added to 5 grams of the polymer solution. The resultant mixture was impregnated into a 4 cm×5 cm polyester nonwoven fabric (RB-273-40-W/R supplied by WPT Non Wovens). The resultant material was dried in a vacuum oven with 0.5 L/min air bleed.

### Example 4

Polyvinyl pyrrolidone 40,000 MW was dissolved in methanol to form a 30% w/v solution. 0.7 grams Sodium Percarbonate, 0.3 g of Super Low Molecular Weight Hyaluronic Acid, 0.3 grams High Molecular Weight Hyaluronic Acid and 0.3 grams of Aloe Vera were added to 5 grams of the polymer solution. The resultant mixture was impregnated into a 4 cm×4 cm polyester nonwoven fabric (RB-273-40-W/R supplied by WPT Non Wovens). The resultant material was dried in a vacuum oven with 0.5 L/min air bleed.

### Example 5

Polycaprolactone was dissolved in acetone to form a 10% w/v solution. 1 gram of Calcium Peroxide was added to 4 grams of the polymer solution. The resultant mixture was impregnated into a 4 cm×4 cm polyester nonwoven fabric (RB-273-40-W/R supplied by WPT Non Wovens). The resultant material was dried in a vacuum oven with 0.5 L/min air bleed.

Measurement of Oxygen Generation

Oxygen generation was determined by placing a 2 cm² sample in a 40 mL Thermo Scientific glass vial filled with RO (reverse osmosis) purified water. A PreSens Microx 4 oxygen sensor housed in a syringe needle was introduced through the septum in the vial lid. The vial was placed in an incubator at 37° C. The probe needle was then pushed through the septum and then the fiber probe pushed out through the needle into the test fluid. The PreSens Microx 4 datalogger was then set to record oxygen levels every minute for the test period.

The results of measurement of oxygen generation for Examples 1 through 5 are shown in FIG. 5. As shown, this technology provides for improved control over the rate of oxygen generation.

### Example 6

A 15% (w/v) polylactic acid (PLA) solution was made in chloroform. 0.5 g of calcium peroxide was added to 7 g of polymer solution, mixed and placed in a 5 ml glass syringe. 7 g of polymer solution without calcium peroxide was placed in a second glass syringe. The glass syringes were placed into syringe pumps and connected to a custom co-axial needle (Ramé-Hart, Succasunna, N.J.). The end of the needle was placed above a glass beaker containing methanol. The syringe pumps were activated to dispense at 0.5 ml/min and the precipitated fiber collected. The fibers were removed from the methanol and dried in a vacuum oven set at 25° C. with a gas flow of 0.5 L/min. The oxygen generating fibers can then be used in formulation of face mask products as desired.

### Example 7

Struts of porcine bone weighing 300 grams were placed in 3000 ml 0.6 M hydrochloric acid for 6 days, with the acid changed every day. After this time the struts were demineralized, as could be confirmed by the ability to bend them by hand. They were rinsed in buffer and stored in a freezer until the next step in the process. A blade with openings 0.030″×0.050″ and a tooth height of 0.012″ was used to produce fibers. The demineralized bone fibers (DBFs) were placed in phosphate buffered saline for 45 minutes.

The demineralized bone fibers weighing 150 grams were placed in 1500 ml of 4 M Guanidine Hydrochloride and placed on a shaker table for 16 hours to remove the bone morphogenic proteins and other naturally present materials.

A 1 mm thick sheet of demineralized bone fibers was made. This was made using a wet lay technique. 15 grams of fibers were suspended in saline to form a slurry and added to a wet lay apparatus having a 4 inch by 4 inch screen. The sheet of fiber on the wet lay screen was removed and placed into a mold that pressed the sheet to a thickness of about 1 mm and then was heated at 50° C. for about an hour with a compression weight placed on top of the fiber sheet. A piece of the resultant sheet 4 cm×4 cm was cut from the sheet.

Polycaprolactone was dissolved in acetone to form a 10% w/v solution. 1 gram of Calcium Peroxide was added to 4 grams of the polymer solution. The resultant mixture was impregnated into the 4 cm×4 cm piece of demineralized bone fiber sheet. The resultant material was dried in a vacuum oven with 0.5 L/min air bleed.

### Example 8

A perfluorocarbon emulsion was made. An emulsifier solutions was made using Polysorbate 20 (Sigma Aldrich) at concentrations of 0.05 gram/ml. An emulsions was made using 3 mls of perfluorodecalin and 2 mls of the emulsifier solutions. Mixing was achieved by rapidly passing the solutions between two syringes joined with a Luer connector. The emulsion was used to activate a piece of the impregnated nonwoven of example 1 by spreading emulsion over its surface.

### Example 9

Polycaprolactone was dissolved in dichloromethane to form a 15% w/v solution. 0.76 grams of Sodium Peroxide was added to 2.23 grams of the polymer solution. The mixture was dried in a vacuum oven with 0.5 L/min air bleed, and then ground to form a powder.

### Example 10

Polycaprolactone was dissolved in acetone to form a 5% w/v solution. 1.15 grams of Sodium perborate was added to 4.5 grams of the polymer solution. The mixture was dried in a vacuum oven with 0.5 L/min air bleed, and then ground to form a powder.

Measurement of Oxygen Generation

Oxygen generation of samples from Examples 9 and 10 was determined by placing 0.1 g of oxygen generator powder sample in a 40 mL Thermo Scientific glass vial filled with RO (reverse osmosis) purified water. A PreSens Microx 4 oxygen sensor housed in a syringe needle was introduced through the septum in the vial lid. The vial was placed in an incubator at 37° C. The probe needle was then pushed through the septum and then the fiber probe pushed out through the needle into the test fluid. The PreSens Microx 4 datalogger was then set to record oxygen levels every minute for the test period.

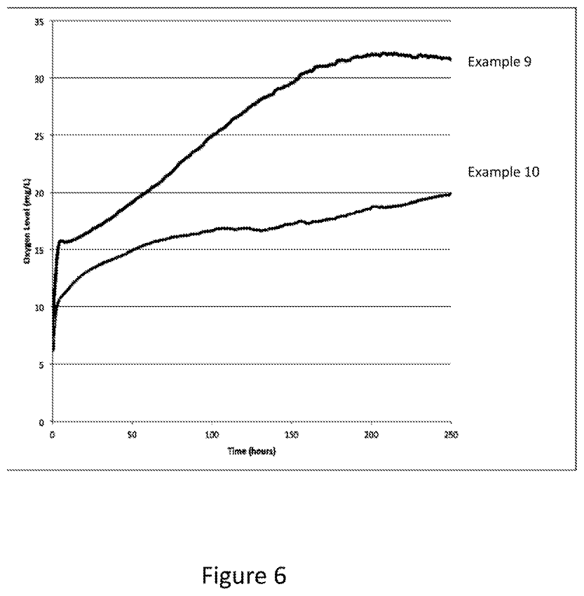

The results of measurement of oxygen generation for Examples 9 and 10 are shown in FIG. 6. As shown, this technology provides for improved control over the rate of oxygen generation, and in these instances, the material was designed to provide sustained oxygen generation for periods of up to 10 days.

While certain embodiments of the present disclosure have been illustrated and described, it is understood by those of ordinary skill in the art that certain modifications and changes can be made to the described embodiments without departing from the spirit and scope of the present disclosure as defined by the following claims, and equivalents thereof.

What is claimed is:

1. A composition for healing or improving skin, the composition comprising:
a fabric; and
an oxygen generating material impregnated in the fabric.

2. The composition of claim 1, further comprising a binding agent.

3. The composition of any of claims 1-2, further comprising a perfluorocarbon or an emulsion comprising a perfluorocarbon.

4. The composition of any of claims 1-3, wherein the fabric is a woven or non-woven material.

5. The composition of claim 4, wherein the woven material is knitted or is a foam.

6. The composition of claim 4, wherein the nonwoven material is a polymer or demineralized bone fibers.

7. The composition of claim 6, wherein the polymer is polyester.

8. The composition of claim 1, wherein the oxygen generating material is calcium peroxide, magnesium peroxide, sodium percarbonate, and/or sodium peroxide.

9. The composition of any of claims 1-8, further comprising a resorbable polymer that encapsulates the oxygen generating material.

10. The composition of any of claims 1-9, further comprising an oxygen barrier backing layer attached to the fabric.

11. The composition of any of claims 1-10, further comprising an additive selected from pentapeptides, copper peptides, calming agents, caffeine, horse chestnut extract, pepper seed extract, arnica flower extract, tranexamic acid, shea butter, sodium hyaluronate, ester of vitamin C, camelia oleifera, retinyl palmitate, sodium bicarbonate, or any combination thereof.

12. The composition of any of claims 1-11, wherein the fabric forms a face mask.

13. A method of delivering oxygen to an area skin, the method comprising applying the composition of claim 1 to the area of skin.

14. The method of claim 13, wherein the area of skin is a face, a wound, a superficial wound, a burn, or acne.

15. A composition for healing or improving skin, the composition comprising:
a heat barrier; and
an oxygen generating material impregnated in the fabric.

16. The composition of claim 15, further comprising a binding agent.

17. The composition of any of claims 15-16, further comprising a perfluorocarbon or perfluorocarbon emulsion.

18. The composition of any of claims 15-17, wherein the heat barrier is a woven or non-woven material.

19. The composition of claim 18, wherein the woven material is knitted or is a foam.

20. The composition of claim 18, wherein the nonwoven material is a polymer or demineralized bone fibers.

21. The composition of claim 20, wherein the polymer is polyester.

22. The composition of claim 15, wherein the oxygen generating material is calcium peroxide, magnesium peroxide, sodium percarbonate, and/or sodium peroxide.

23. The composition of any of claims 15-22, further comprising a resorbable polymer that encapsulates the oxygen generating material.

24. The composition of any of claims 15-23, further comprising an oxygen barrier backing layer attached to the heat barrier.

25. The composition of any of claims 15-24, further comprising an additive selected from pentapeptides, copper peptides, calming agents, caffeine, horse chestnut extract, pepper seed extract, arnica flower extract, tranexamic acid, shea butter, sodium hyaluronate, ester of vitamin C, camelia oleifera, retinyl palmitate, sodium bicarbonate, or any combination thereof.

26. The composition of any of claims 15-25, wherein the heat barrier forms a face mask.

27. A method of delivering oxygen to an area of skin, the method comprising applying the composition of claim 15 to the area of skin.

28. The method of claim 27, wherein the area of skin is a face, a wound, a superficial wound, a burn, or acne.
